(19) United States
(12) Patent Application Publication
(10) Pub. No.: US 2017/0072168 A1
(43) Pub. Date: Mar. 16, 2017

(54) GRAPHIC FOR MEDICAL ARTICLE SECUREMENT SYSTEMS AND METHODS OF USING SAME

(71) Applicant: 3M INNOVATIVE PROPERTIES COMPANY, SAINT PAUL, MN (US)

(72) Inventors: NAIMUL KARIM, MAPLEWOOD, MN (US); SHILPI K. SANGHI, SAINT PAUL, MN (US); JENNIFER N. HANSON, SAINT PAUL, MN (US); PETER M. EISENBERG, MINNEAPOLIS, MN (US); JIA HU, MOUNDS VIEW, MN (US); THOMAS G. SKULLEY, SAINT PAUL, MN (US)

(73) Assignee: 3M INNOVATIVE PROPERTIES COMPANY, SAINT PAUL, MN (US)

(21) Appl. No.: 15/122,969

(22) PCT Filed: Feb. 25, 2015
(86) PCT No.: PCT/US2015/017495

§ 371 Date: Sep. 1, 2016

Related U.S. Application Data
(60) Provisional application No. 61/948,142, filed on Mar. 5, 2014.

(51) Int. Cl. A61M 25/02

(52) U.S. Cl. CPC A61M 25/02; A61M 2205/58; A61M 2025/024; A61M 2025/0266

(57) ABSTRACT

A graphic for use with medical article securement systems, medical article securement systems comprising a graphic, and methods of using same. The graphic can include a representation of an anatomical feature, such that the graphic provides a visual cue for coupling the medical article securement system to a subject in a desired orientation relative to the subject. Medical article securement systems comprising a graphic can be configured to be coupled to the subject and further configured to secure at least a portion of a medical article. Methods can include coupling the medical article securement system to the subject according to the graphic.

### FIELD

The present disclosure generally relates to a graphic for use with medical article securement systems and methods, and medical article securement systems and methods comprising the graphic, for securing a medical article to a subject, and particularly, for securing various catheter systems, tubes, or other elongated devices to the subject.

### BACKGROUND

In various medical treatments, it can be necessary to secure a medical article to a subject for various lengths of time, e.g., with a securement device, or medical article securement system. In some cases, such securement devices can be first attached to the subject, followed by securing the medical article to the securement device. In such cases, since most securement devices are designed to couple with a medical article in a very specific way, it can be important that the device be coupled to the subject in a very specific orientation, relative to the subject's anatomy. Even in situations where a securement device is first coupled with a medical article, prior to its attachment to the subject, it can be helpful to know how the securement device is to be oriented relative to the patient anatomy. In either case, a clear visual cue as to the required orientation of a securement device, relative to the subject, can be advantageous for a quick, easy and failure-free securement, especially for unskilled users, if the user is under time pressure, and/or if the user is not familiar with a specific securement device.

### SUMMARY

The present disclosure is generally directed to a graphic for use with medical article securement systems and methods, medical article securement systems and methods comprising a graphic, methods for securing medical articles to medical article securement systems. Such a graphic generally represents or mimics an anatomical feature (i.e., of a subject of interest, such as a human), such that the graphic provides a visual cue (i.e., guidance) for coupling the medical article securement system (and, ultimately, the medical article) to the subject (e.g., a living subject) in a desired (e.g., therapeutic) orientation relative to the subject. The medical article securement systems and methods of the present disclosure are generally designed to facilitate coupling and decoupling a medical article to and from the system, while also providing means for reliably retaining or securing a medical article, e.g., a catheter system, relative to a subject, for a desired treatment period.

One aspect of the present disclosure provides a graphic for use with a medical article securement system. The graphic can include a representation of an anatomical feature, such that the graphic provides a visual cue for coupling the medical article securement system to a subject in a desired orientation relative to the subject.

Another aspect of the present disclosure provides an article comprising a graphic of the present disclosure, and a backing. The backing can have a first side and a second side opposite the first side, and the second side of the backing can be configured to be coupled to a medical article securement system. The graphic can be provided on at least one of the first side and the second side of the backing.

Another aspect of the present disclosure provides a medical article securement system. The system can include a graphic comprising a representation of an anatomical feature, such that the graphic provides a visual cue for coupling the medical article securement system to a subject in a desired orientation relative to the subject. The system can be configured to be coupled to the subject and further configured to secure at least a portion of a medical article.

Another aspect of the present disclosure provides a method for coupling a medical article securement system to a subject. The method can include providing a medical article securement system comprising a graphic. The graphic can include a representation of an anatomical feature, such that the graphic provides a visual cue for coupling the medical article securement system to a subject in a desired orientation relative to the subject. The method can further include coupling the medical article securement system to the subject according to the graphic.

Another aspect of the present disclosure provides a medical article securement system. The system can include a base dressing configured to be coupled to a subject, a bracket coupled to the base dressing, and a graphic comprising a representation of an anatomical feature. The bracket can be configured to secure at least a portion of a medical article.

Another aspect of the present disclosure provides a method for coupling a medical article securement system to a subject. The method can include providing a medical article securement system. The system can include a base dressing configured to be coupled to a subject, a bracket coupled to the base dressing, and a graphic comprising a representation of an anatomical feature. The bracket can be configured to secure at least a portion of a medical article. The method can further include coupling the medical article securement system to the subject according to the graphic.

Other features and aspects of the present disclosure will become apparent by consideration of the detailed description and accompanying drawings.

### BRIEF DESCRIPTION OF THE DRAWINGS

FIG. 1 is a perspective view of a medical article securement system according to one embodiment of the present disclosure, showing a medical article coupled to the medical article securement system and the medical article securement system coupled to a subject, the medical article securement system comprising a bracket, a base dressing, and a graphic.

FIG. 1A is a perspective view of a medical article securement system according to another embodiment of the present disclosure, showing a medical article coupled to the medical article securement system and the medical article securement system coupled to a subject.

FIG. 2 is a perspective view of the medical article securement system of FIG. 1, shown without the medical article, prior to coupling to a subject.

FIG. 3 is a top plan view of the medical article securement system of FIGS. 1 and 2.

FIG. 4 is a bottom plan view of the medical article securement system of FIGS. 1, 2 and 3, showing alternative locations for the graphic.

FIG. 4A is a perspective view of a medical article securement system according to another embodiment of the present disclosure.

FIG. 5 is a medical article securement system according to another embodiment of the present disclosure, including a graphic according to another embodiment of the present disclosure.

FIG. 6 is a perspective view of an article according to one embodiment of the present disclosure, the article comprising a graphic provided in a roll.

FIG. 7 is a top plan view of a medical article securement system according to another embodiment of the present disclosure, including a graphic according to another embodiment of the present disclosure.

FIG. 8 is a perspective view of a medical article securement system according to another embodiment of the present disclosure, including a graphic according to another embodiment of the present disclosure.

FIG. 9 is a perspective view of a medical article securement system according to another embodiment of the present disclosure, including a graphic according to another embodiment of the present disclosure.

FIG. 10 is a perspective view of a medical article securement system according to another embodiment of the present disclosure, including a graphic according to another embodiment of the present disclosure.

FIG. 11 is a perspective view of a medical article securement system according to another embodiment of the present disclosure, including a graphic according to another embodiment of the present disclosure.

### DETAILED DESCRIPTION

Before any embodiments of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “coupled,” and variations thereof are used broadly and encompass both direct and indirect mountings and couplings. It is to be understood that other embodiments may be utilized, and structural or logical changes may be made without departing from the scope of the present disclosure. Furthermore, terms such as “front,” “back,” and the like are only used to describe elements as they relate to one another, but are in no way meant to recite specific orientations of the apparatus, to indicate or imply necessary or required orientations of the apparatus, or to specify how the invention described herein will be used, mounted, displayed, or positioned in use.

The present disclosure generally relates to graphics for use with medical article securement systems and methods, and medical article securement systems and methods comprising one or more graphics, for safely and reliably securing a medical article, such as a catheter system, upon a desired location of a subject's body (e.g., on the subject's skin). The medical article securement systems can be universal to accommodate and reliably secure a large variety of medical articles or class of medical articles (e.g., catheters), and can be particularly useful for securing medical articles that need to be secured to a subject over a prolonged period of time, such as weeks or months. Medical article securement systems of the present disclosure are particularly suited for being coupled to the subject and are further configured to secure at least a portion of a medical article, i.e., to inhibit movement of the medical article in at least one of a longitudinal direction, a lateral direction, and a normal direction, relative to the medical article securement system and/or the subject to which the medical article securement system is coupled.

The graphic can generally include a representation of an anatomical feature of a subject to which the medical article securement system is to be coupled. As a result of the positioning and/or orientation of the anatomical feature, the graphic can provide a visual cue for coupling the medical article securement system (and, ultimately, the medical article) to the subject in a desired, safe, proper, or therapeutic orientation, relative to the subject.

Examples of medical articles that can be employed with the medical article securement system of the present disclosure include, but are not limited to, connector fittings; catheter systems (e.g., including catheters, catheter hubs, catheter adaptors, etc.); fluid supply lines; medical tubes (e.g., gastric feeding or drainage tubes (G tubes), endotracheal tubes, nasogastric tubes (NG tubes), or the like); other similar articles; or combinations thereof. Examples of catheter systems can include, but are not limited to, urinary catheters, intravenous (IV) catheters, central venous catheters (CVCs), peripherally inserted central catheters (PICCs), midline intravenous catheters, implanted ports, arterial catheters, and dialysis catheters.

The terms “longitudinal” and “axial” are used to refer to a direction or axis that is generally parallel to the direction in which the medical article extends and generally parallel to the overall direction of fluid flow, e.g., along a catheter line.

The term “lateral” is used to refer to a direction or axis that is perpendicular to the longitudinal axis or direction and is used to represent side-to-side motion of a medical article.

The terms “vertical” and “normal” are used interchangeably to refer to a direction or axis that is normal to both the longitudinal and lateral directions or axes, as well as to the surface of a subject's skin (or of the medical article securement system) when the medical article securement system is coupled to the subject's skin, and is used to represent the direction of motion toward and away from the skin surface.

The term “proximal” and “distal” are used to represent axial directions, relative to a medical practitioner operating or holding the medical article. That is, the term “distal” is used to refer to the direction away from the medical practitioner (e.g., toward an insertion site of the medical article on the subject and inside the subject's body), and the term “proximal” is used to refer to the direction toward the medical practitioner (e.g., toward the outside of the subject's body, away from an insertion site). For example, the distal end of a catheter is inserted into the subject, while the proximal end extends exterior of the subject toward the medical practitioner. The distal end of the medical article securement system refers to the end of the system that is configured to be oriented toward the distal end of the medical article to which it will be coupled, and the proximal end of the medical article securement system refers to the end of the system that is configured to be oriented toward the proximal end of the medical article. As a result, in the case of catheter systems, the distal end of the medical article securement system will be oriented toward the insertion site on the subject's body, and the proximal end of the of the medical article securement system will be oriented away from the insertion site on the subject's body.

The phrase “substantially transparent” is generally used to refer to a body, substrate or material that transmits at least 50% of electromagnetic radiation having wavelengths at a selected wavelength or within a selected range of wavelengths in the ultraviolet to infrared spectrum (e.g., from about 200 nm to about 1400 nm; “UV-IR”), in some embodiments, at least about 75% of a selected wavelength (or range) in the UV-IR spectrum, and in some embodiments, at least about 90% of a selected wavelength (or range) in the UV-IR spectrum.

The phrase “substantially non-transparent” is generally used to refer to a body, substrate or material that transmits less than 50% of electromagnetic radiation having wavelengths at a selected wavelength or within a selected range of wavelengths in the ultraviolet to infrared spectrum (e.g., from about 200 nm to about 1400 nm; “UV-IR”), in some embodiments, less than 25% of a selected wavelength (or range) in the UV-IR spectrum, and in some embodiments, less than 10% of a selected wavelength (or range) in the UV-IR spectrum.

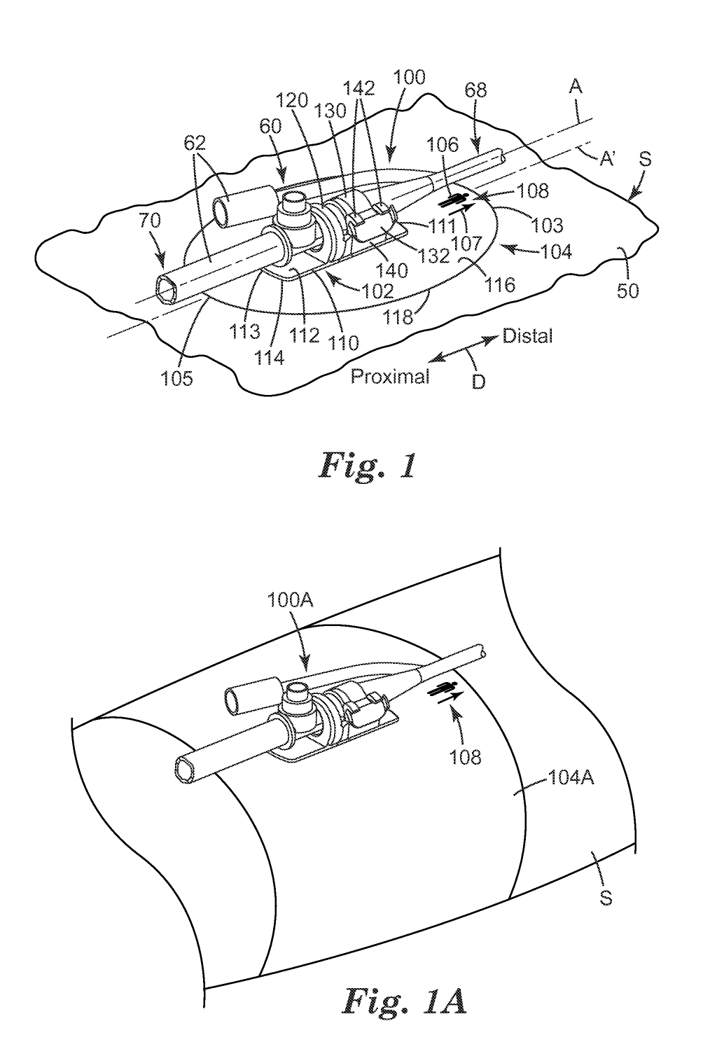

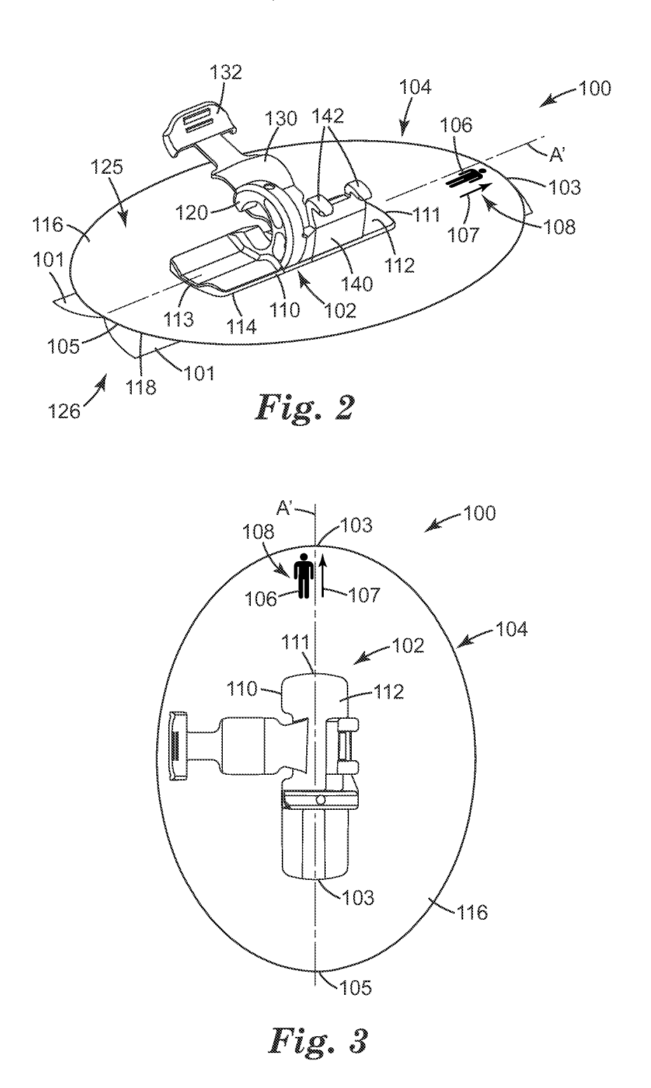

FIGS. 1 and 2-4 illustrate a medical article securement system 100 and graphic 108 according to one embodiment of the present disclosure. FIG. 1 illustrates a system 100 coupled to the skin 50 of a subject S, e.g., near an insertion site of interest for a catheter system. FIG. 1 also illustrates an exemplary medical article 60 coupled to the system 100. By way of example only, the medical article 60 is illustrated as being a catheter system, and particularly, a urinary catheter system, having one or more catheters (or tubes or lines) 62, and a catheter hub 64. FIG. 2 shows the system 100 prior to coupling the system 100 to the subject.

As shown, the medical article 60 can have a longitudinal axis A that extends along and defines a longitudinal direction D. The medical article can extend distally to include a first longitudinal, distal end or portion 68 (which may extend beyond what is shown in FIG. 1, e.g., into an interior of the subject's body), and can extend proximally to include a second longitudinal, proximal end or portion 70 (which may extend further proximally and include additional elements than what is shown in FIG. 1). Accordingly, the system 100 (and/or its components) can include a first longitudinal, distal end (e.g., to be oriented toward an insertion site), and can a second longitudinal, proximal end (e.g., to be oriented away from an insertion site).

As shown in FIGS. 1-4, the system 100 can include a bracket (or “retaining bracket” or “retainer” or “medical article attachment mechanism”) 102, a base dressing 104, and the graphic 108. The bracket 102 and other components of the system 100 can be coupled to the base dressing 104, and the base dressing 104 can be adhered to the skin 50. The graphic 108, which is described in greater detail below, can be configured to represent or mimic an anatomical feature of the subject S to provide a visual cue for orienting the medical article 60 relative to the system 100 (and/or the subject S) and/or for orienting the system 100 relative to the subject S. The graphic 108 can be coupled to or provided by (e.g., integrally formed with) the bracket 102, the base dressing 104, or another portion of the system 100, as described in greater detail below with respect to other figures of the present disclosure.

The visual cue provided by the graphic 108 is generally a simple, easy-to-follow (e.g., for non-skilled users) visual guide to allow for the medical article securement system to be properly aligned or positioned on the subject, e.g., relative to the pertinent anatomy. The representation of the anatomy or anatomical feature can be realistic (e.g., anatomically-accurate), can include an artistic representation (e.g., a cartoon, an outline, a geometrical representation, etc.), can include other suitable graphic representations, or a combination thereof.

The anatomical feature that is represented can be at least a portion of an anatomy, including, but not limited to a whole body, an upper body (e.g., above the waist), a lower body (e.g., below the waist), a body part, an organ, or a combination thereof.

In some embodiments, the anatomical feature can include a body part that is visible on the subject (i.e., an external feature, such as an ear, hand or body outline) or a body part that is not visible on the subject (i.e., an internal feature, such as a heart, brain, stomach, etc.). Examples of anatomical features that can be represented by the graphic 108 can include, but are not limited to, at least a portion of at least one of a limb, a hand, a foot, a finger, a toe, a head, an eye, an ear, a nose, a mouth, teeth, a neck, a torso, an internal organ, and a combination thereof. By way of further example, in some embodiments, the anatomical feature can include at least a portion of at least one of a heart, a stomach, a bladder, a kidney, a brain, a lung, a skeletal feature (e.g. spine, femur, rib cage, etc.), another suitable organ or feature, and a combination thereof.

While FIG. 1 illustrates a base dressing 104 comprising a patch configuration that can be adhesively coupled to the subject S, FIG. 1A illustrates a medical article securement system 100A according to another embodiment of the present disclosure that includes all of the features of the medical article securement system 100, including the graphic 108, except that the medical article securement system 100A includes a base dressing 104A that is configured to be mechanically coupled to the subject S, e.g., by being wrapped around a limb, as illustrated. In such embodiments, the base dressing 104A can be in the form of a wrap (e.g., a compression wrap, a non-compressive flexible band), a wound dressing, a bandage, or the like, or combinations thereof. The base dressing 104A can include or be used in combination with any necessary fasteners, such as hooks, adhesives, hook-and-loop fasteners, or the like, or combinations thereof. Such fasteners can be used, for example, to secure an overlapping region of the base dressing 104A after it has been coupled to the subject S (e.g., around a body part).

The system 100A and the base dressing 104A are illustrated by way of example only. It should be understood that any further description of the use or employment of a base dressing in systems of the present disclosure (e.g., the types of coupling between the bracket 102 and the base dressing 104, the various means for applying a graphic to the base dressing 104, etc.) can also be applied to the system 100A and the base dressing 104A.

With continued reference to the embodiment of FIGS. 1 and 2-4, the bracket 102 can include a base (or “platform”) 110. The base 110 (or the bracket 102 or the system 100) can include a longitudinal axis A′ that is oriented along or parallel to the longitudinal axis A of the medical article 60 when the medical article 60 is coupled to the bracket 102. That is, when the medical article 60 is coupled to the bracket 102, the longitudinal axis A of the medical article 60 can be generally aligned with (which can include directly overlapping or just parallel to) the longitudinal axis A′ of the base 110. The longitudinal axis A′ of the base 110 (or the bracket 102) also extends along or defines the longitudinal direction D.

The base 110 of the bracket 102 can include a first major surface 112 (e.g., an upper surface) configured to face away from the subject's skin 50 and to receive at least a portion of the medical article 60. The base 110 can further include a second major surface 114 (e.g., a bottom surface) opposite the first major surface 112 that is configured to face the subject's skin 50 and to be securely coupled (e.g., adhered) to the base dressing 104. The base 110 (or the bracket 102) can further include a distal end (or first longitudinal end) 111 and a proximal end (or second longitudinal end) 113, such that, for example, the distal end 111 is configured to be positioned away from a medical practitioner operating or holding the medical article 60, and the proximal end 113 is configured to be positioned toward the medical practitioner. In the example of a catheter system 60, as shown, the distal end 111 of the base 110 can be positioned toward an insertion site of the catheter, and the proximal end 113 of the base 110 can be positioned away from the insertion site. The longitudinal axis A′ can extend in the longitudinal direction (e.g., as defined by the medical article 60) between the distal end 111 and the proximal end 113.

By way of example only, the same bracket 102 is illustrated in all of the figures of the present disclosure and is particularly suited for retaining urinary catheters. The bracket is generally a rigid structural element capable of securing the medical article of interest and may include one or more posts, arms and/or movable members configured to inhibit movement of the medical article in at least one of a longitudinal direction, a lateral direction, and a normal direction. The bracket 102 is generally used to provide an element that is more rigid than the base dressing 104 (or base dressing 104A) that can securely and reliably retain the medical article 60. However, it should be understood that the illustrated bracket 102 is merely representative of one type of rigid bracket that can be employed in medical article securement systems of the present disclosure and is not intended to be limiting. By way of example, the bracket 102 is illustrated as being generally centrally located on the base dressing 104; however, this need not be the case. Additional details of the illustrated embodiment of the illustrated bracket 102 will now be described.

As shown in FIGS. 1 and 2, the illustrated bracket 102 includes a first arm 120 that is rigidly fixed to (e.g., integrally formed with) the base 110 of the bracket 120. The first arm 120 can be configured to retain a portion of the medical article 60 underneath it. The first arm 120 can therefore serve to inhibit movement of the medical article 60 primarily in a normal direction. However, due to the shape and configuration of the first arm 120, the first arm 120 can additionally inhibit movement of the medical article 60 in a lateral and/or longitudinal direction. For example, as shown in FIG. 1, in some embodiments, the first arm 120 can be configured to extend upwardly from the base 110 in a normal direction and can function as a longitudinal stop for at least a portion of the medical article 60.

As further illustrated, the bracket 102 includes a second arm 130 that can be coupled to or integrally formed with the base 110. The second arm 130 of the illustrated embodiment is flexible and can be movable (e.g., via one or more hinges) between a first, open position (see, e.g., FIG. 2) and a second, closed (or latched) position (see, e.g., FIG. 1). As shown, the bracket 102 can further include one or more supports 140 that are rigidly fixed to (e.g., integrally formed with) the base 110, and which can serve as an engaging element for the second arm 130 to allow the second arm 130 to be latched in its second position. Particularly, in the illustrated embodiment, the second arm 130 can include a terminal flange 132 and the support 140 can include one or more hooks 142. The terminal flange 132 can be configured to be received under the one or more hooks 142 of the support 140 to latch or lock the second arm 130 in its second position. As a result, the support 140 can inhibit the medical article 60 from lateral movement, and the second arm 130 can inhibit the medical article 60 from movement in the lateral and normal directions. In addition, if the medical article 60 were to include any elements that protrude above the vertical position of the second arm 130 when in its closed position, the second arm 130 may also inhibit movement of the medical article 60 in a longitudinal direction.

While the rigid, mechanical bracket 102 is illustrated by way of example only, systems of the present disclosure can additionally or alternatively include one or more flexible flaps or strips (e.g., comprising a hook-and-loop fastener, an adhesive, or combinations thereof) that can also be coupled to or integrally formed with the base dressing 104 (or the base dressing 104A). Such flaps can be configured to (but need not) interact with bifurcating or protruding features of a medical article for securement. In embodiments employing adhesive flaps, for example, securement of a medical article can rely partially or wholly on adhesion of the flaps to or about the medical article.

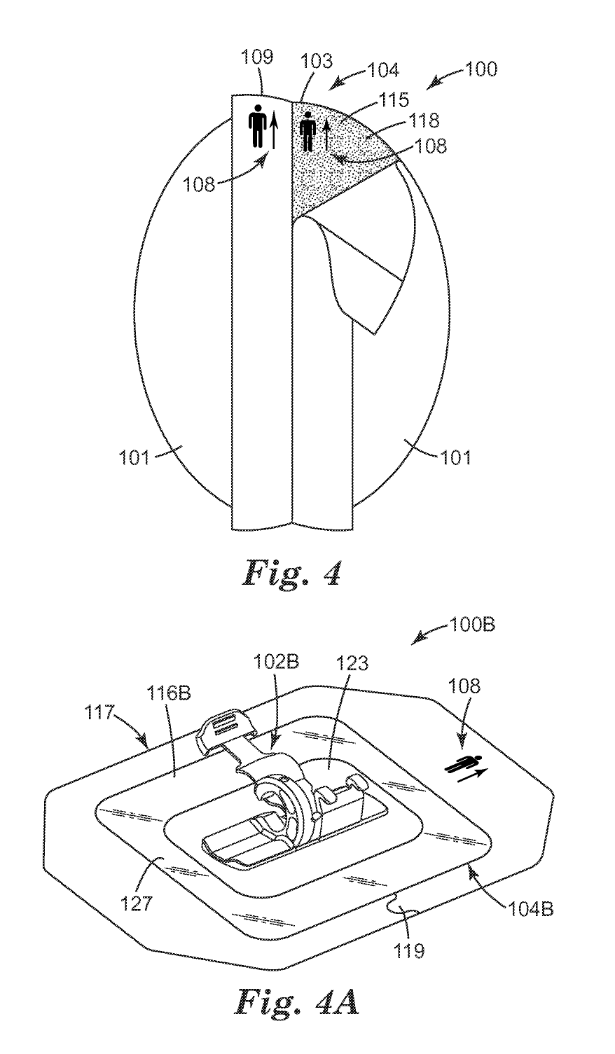

In some embodiments, the base dressing 104 can include a distal end (or first longitudinal end) 103 and a proximal end (or second longitudinal end) 105. The base dressing 104 can further include a first side 116 configured to face away from the subject's skin 50, and a second side 118 opposite the first side 116 that comprises a skin-contact adhesive 115 (see FIG. 4) for adhering to the skin 50. The second major surface 114 is configured to be coupled to the first side 116 of the base dressing 104. Although only a single shape of the base dressing 104 is illustrated in FIGS. 1 and 2-4, it should be understood that the base dressing 104 can take on a variety of shapes and sizes. Such shapes can depend on the shapes and configurations of the other elements of the system 100 and the medical article 60, and whether the shape of the base dressing 104 will form any portion of the graphic 108 (see, e.g., FIGS. 8 and 10, described in greater detail below). In some embodiments, the base dressing 104 comprises a laminated structure comprising one or more layers of a fabric, a woven fibrous web, a nonwoven fibrous web, a knit, a polymeric film, a polymeric foam, or combinations thereof. In some embodiments, at least a portion of the base dressing 104 can be substantially transparent.

The skin-contact adhesive 115, when employed, is generally a pressure-sensitive adhesive, and particularly is a pressure-sensitive adhesive that is capable of securely but releasably adhering or bonding to skin (e.g., mammalian skin). The skin-contact adhesive 115 is also generally safe and non-toxic. Skin-contact adhesive layers will generally be selected according to the desired end use of the base dressing 104. In some embodiments, the base dressing 104 can include more than one skin-contact adhesive 115. Where the base dressing 104 comprises more than one skin-contact adhesive layer 115, each skin-contact adhesive layer 115 may be selected independently of each other with regard to material and thickness used. Examples of suitable adhesives include acrylates, silicones, polyisobutylenes, synthetic rubber, natural rubber, and copolymers and mixtures thereof. Acrylates and silicones can be preferred skin-contact adhesives 115. In general, the skin-contact adhesive 115 should cause little or no irritation or sensitization of the skin during the intended wear period. Examples of skin-contact adhesives 115 that can be employed with the systems of the present disclosure include, but are not limited to, the adhesives described in U.S. Pat. No. RE24,906; 3,389,827; 6,103,369 and 4,499,896, which are incorporated herein by reference. In addition, silicone adhesives such as those described in U.S. Patent Publication No. 2011/0212325, which is incorporated herein by reference, can also be employed.

In some embodiments, e.g., in embodiments employing silicone adhesives, the base dressing 104 and the skin-contact adhesive 115 can be perforated to provide openings from the first side 116 of the base dressing 104 all the way through the second side 118 and the skin-contact adhesive 115, which can enhance permeability of the base dressing 104 and can minimize moisture build-up at the skin surface underlying the base dressing 104.

As shown in FIGS. 2 and 4, in some embodiments, the system 100 can further include one or more release liners 101 that can provide a release layer or surface to the skin-contact adhesive 115 on the second side 118 of the base dressing 104 prior to use. By way of example only, as clearly shown in FIG. 2, the system 100 includes two butterfly-configured release liners 101, such that one portion (e.g., one lateral half) of the base dressing 104 can be applied at a time to the subject's skin 50 to facilitate adhering the system 100 to the skin 50 in a desired orientation without any crinkling or folding of the base dressing 104 before it is ready to be applied. The release liners 101 are illustrated as being symmetrical, however, this need not be the case, depending on the shape and configuration of the base dressing 104.

Examples of liners suitable for use with systems of the present disclosure can include, but are not limited to, kraft papers, polyethylene, polypropylene, polyester, or combinations thereof. Such liners can be coated with release agents, such as fluorochemicals, silicones, or other suitable low surface energy materials. Other adhesives and release liner 101 combinations known to those of ordinary skill in the art can be employed in the systems of the present disclosure.

The bracket 102 (e.g., the second major surface 114 of the base 110) can be coupled to the base dressing 104. In some embodiments, the bracket 102 can be permanently coupled to the base dressing 104 (or the base dressing 104A of FIG. 1A), e.g., by coupling means that, if undone or decoupled, would result in irreparable damage to the system 100, such that the system 100 could no longer be used for its intended purposes. For example, in some embodiments, the bracket 102 can be permanently coupled to the base dressing 104 by any of a variety of permanent coupling means, including, but not limited to, adhesives, stitches, staples, screws, nails, rivets, brads, crimps, welding (e.g., sonic (e.g., ultrasonic) welding), any thermal bonding technique (e.g., heat and/or pressure applied to one or both of the components to be coupled), snap-fit engagement, press-fit engagement, heat sealing, other suitable permanent or semi-permanent coupling means, and combinations thereof. In some embodiments, the bracket 102 can be removably coupled to the base dressing 104 (or the base dressing 104A of FIG. 1A), e.g., by coupling means that allow for the bracket 102 and the base dressing 104 to be decoupled without damage to either the bracket 102 or the base dressing 104. Such removable coupling means can include, but are not limited to, screw threads or other inter-engaging features; press-fit engagement (also sometimes referred to as “friction-fit engagement” or “interference-fit engagement”); snap-fit engagement; magnets; hook-and-loop fasteners; adhesives; cohesive; clamps; other suitable removable coupling means, and combinations thereof.

In embodiments in which the bracket 102 and the base dressing 104 are removably coupled to one another, the graphic 108 can further provide a visual cue for orienting the bracket 102 with respect to, and/or coupling the bracket 102 to, the base dressing 104.

In some embodiments, the system 100 can include a first, front side 125 and a second, back side 126 opposite the first side (see FIG. 2), where the first side 125 is positioned to face away from the subject S when the system 100 is coupled to the subject S. In some embodiments, the first side 125 can be provided or defined by at least one of the base dressing 104, the bracket 102 and any disposable portion (e.g., a carrier or frame). The bracket 102 can be configured to inhibit movement of the medical article 60 in at least one of a longitudinal direction, a lateral direction, and a normal direction, relative to the first side 125. In some embodiments, the second side 126 can also be provided by at least one of the base dressing 104, the bracket 102 and any disposable portion (e.g., the release liners 101). The graphic 108 can be provided by or visible from the first side 125 and/or the second side 126 of the system 100. For example, in some embodiments, the graphic 108 can be provided on the first surface 112 and/or the second surface 114 of the bracket 102, or somewhere in between (e.g., embedded intermediately of the first surface 112 and the second surface 114). In addition, in some embodiments, the graphic 108 can be provided on the first surface 116 and/or the second surface 118 of the base dressing 104, or somewhere in between (e.g., embedded intermediately of the first surface 116 and the second surface 118). If the bracket 102 is substantially transparent, the graphic 108 can be visible from the first side 125 and/or the second side 126 of the system 100, no matter where (e.g., within or on the bracket 102) the graphic 108 is located. Also, in some embodiments, the second surface 114 of the bracket 102 can be exposed on the second side 126 of the system 100. Similarly, if the base dressing 104 is substantially transparent, the graphic 108 can be visible from the first side 125 and/or the second side 126 of the system 100, no matter where (e.g., within or on the base dressing 104) the graphic 108 is located. As a result, the graphic 108 can be provided on one or more of a variety of surfaces of the system 100 so as to be visible on the first side 125 and/or the second side 126 of the system 100.

With continued reference to FIGS. 1 and 2-4, by way of example, the first feature 106 of FIGS. 1 and 2-4 includes a representation of an overall human form, or of an entire body. The body, or human figure, is oriented on the system 100 (and particularly, on the base dressing 104 by way of example) such that the head of the figure is oriented toward the distal end of 103 of the base dressing 104.

In some embodiments, the graphic 108 can be located toward (i.e., not centered, but more on one half of the system 100) or adjacent one end of the system 100, e.g., the end that should be pointing toward the anatomy that is represented by the graphic 108.

Positioning of the graphic 108 on the system 100 can therefore provide additional information to a user. By way of example only, the graphic 108 is shown as being located toward, and even adjacent, the distal, longitudinal end 103 of the base dressing 104 to signify to a user that the distal end 103 of the base dressing 104 should be oriented toward the head of the subject S to which the system 100 is to be coupled. However, the graphic 108 can also be located toward one lateral side or end (i.e., not centered laterally, but on at least one lateral half of the system 100) to give additional positioning cues to a user.

In some embodiments, as shown in FIGS. 1 and 2-5, the graphic 108 can include two features—a first feature 106 that includes a representation of an anatomical feature, and a second feature 107 that is non-anatomical but gives additional information to a user for properly coupling the medical article 60 to the system 100 and/or for coupling the medical article 60 and/or the system 100 to the subject 100.

Not all embodiments of the graphic 108 of the present disclosure include more than one feature. Some embodiments include only a representation of an anatomical feature; however, the addition of a second (e.g., directional) feature 107 can further indicate the proper orientation of the system 100 to a user.

By way of further example, the second feature 107 includes a directional cue, and particularly, an arrow. As shown in FIGS. 1 and 2-3, the arrow is pointing toward the distal end 103 of the base dressing 104 and in the same direction as the head of the figure of the first feature 106 of the graphic 108, further reinforcing to a user that the distal end 103 of the base dressing 104 should point toward the head of the subject S when coupling the system 100 to the subject S. The second feature 107 (the arrow) is also sized to have the same length in the longitudinal direction as the first feature 106 (the figure), although this need not be the case.

Other examples of directional cues that can be employed include, but are not limited to, an open umbrella, an outline of a house or but (e.g., with the tip of the roof indicating the top direction), or a combination thereof.

As mentioned above, the first (anatomical) feature 106 of the graphic 108 is in the shape of a body or a figure that mimics or represents that of the subject S to which the system 100 is configured to be coupled. As a result, if the system 100 is configured for a non-human (e.g., animal) subject, the figure in the graphic 108 can be configured accordingly.

The body (i.e., the figure) shape or outline is representative, and is not precisely anatomically accurate. Rather, the outline is representative of anatomy and includes sufficient information or detail to allow a user to quickly and easily ascertain what is being represented so as to quickly and easily ascertain how the system 100 needs to be oriented relative to the subject S. That is, the graphic 108, and particularly, the first feature 106, can be configured to provide a visual cue for how to couple the system 100 to the subject S such that the medical article 60 secured to the system 100 is properly oriented relative to the subject S when the system 100 is coupled to the subject S and the medical article 60 is coupled to the system 100.

By way of example only, the graphic 108 is located on the base dressing 104, and particularly, on the exposed, first surface 116 of the base dressing 104. However, as described in greater detail below, the graphic 108 can alternatively, or additionally, be presented on another portion of the system 100, such as the bracket 102, or a disposable portion of the system 100, such as a release liner 101, or a carrier or frame (see, e.g., FIG. 4A, described in greater detail below).

As shown in FIG. 4, in some embodiments, the graphic 108 can alternatively or additionally be provided on one or more release liners 101 (i.e., on the back of the system 100), such that the graphic 108 is visible to a user before and during coupling of the system 100 to the subject S. Additionally, or alternatively, as further shown in FIG. 4, the graphic 108 can be provided on the second surface 118 of the base dressing 104, such that the graphic 108 is visible after at least a portion of a release liner 101 has been removed from the second surface 118. For example, the graphic 108 can be provided on the second surface 118 in a region or area where no skin-contact adhesive 115 is located; the graphic 108 can be provided on the second surface 118 behind the adhesive 115 (e.g., if the adhesive 115 is substantially transparent); and/or the graphic 108 can be provided on or over the adhesive 115. In some embodiments in which the graphic 108 is provided on the second surface 118 of the base dressing 104, the base dressing 104 can be substantially transparent in that region so that the graphic 108 is still visible on the first surface 116 of the base dressing 104, and/or the graphic 108 can be visible on the second surface 118 to a user during application, e.g., during removal of the release liners 101, as shown in FIG. 4. In some embodiments, the graphic 108 can be embedded within the base dressing 104 (or another component of the system 100), e.g., by being laminated between layers thereof, such that the graphic 108 is visible on the first surface 116 and/or the second surface 118 of the base dressing 104.

As further shown in FIG. 4, the graphic 108, even when located on the second surface 118 of the base dressing 104, can be located toward or adjacent one end (e.g., the distal end 103) of the base dressing 104, e.g., the end that should be pointing toward the anatomy that is represented by the graphic 108. In the embodiment shown in FIG. 4, the graphic 108 indicates that the distal end 103 of the base dressing 104 should be oriented toward the head of the subject S.

In addition, when located on a release liner 101, the graphic 108 can be located toward or adjacent one end of the release liner 101. As shown by way of example, the graphic 108 is illustrated in FIG. 4 as being located toward a distal end 109 of the release liner 101.

As a result, multiple graphics 108 can be employed on a single system 100, for example, in different locations. In some embodiments, as shown, the plurality of graphics 108 can be the same, and in some embodiments, the graphics 108 in different locations can be slightly different. In addition, in some embodiments, the first feature 106 and the second feature 107 need not always both be present in any of all of the multiple locations. However, enhanced user confidence and instructional clarity may be achieved by using the same graphic 108 in each location when multiple graphic locations are employed.

The graphic 108 located on the release liner 101 in FIG. 4 illustrates one example of an embodiment in which the graphic 108 is located on a disposable portion of the system 100. FIG. 4A illustrates a medical article securement system 100B according to another embodiment of the present disclosure, in which the graphic 108 is employed on a carrier 117. Particularly, in embodiments such as those shown in FIG. 4A, where a center portion of the carrier 117 is removed, the carrier can function as, and be referred to as, a “frame.” Such carriers or frames can be useful, for example, when a base dressing 104B is employed that is relatively thin and pliable and more likely to wrinkle on application. The carrier 117 can be configured to add integrity and/or rigidity to the base dressing 104B during application. After the base dressing 104B has been applied to a subject, the carrier 117 can then be removed from the first surface 116B of the base dressing 104B, which can be facilitated by one or more tabs 119 on the carrier 117.

The material used to form the carrier 117 is generally substantially more rigid than the base dressing 104B to prevent the base dressing 104B from improperly wrinkling during application to a patient. The carrier 117 can be heat-sealable to the base dressing 104B with or without a low adhesion coating. In some embodiments, a low adhesion coating (low adhesion backsize or LAB) may be provided on the first surface 116B of the base dressing 104B at least in a region that comes in contact with the carrier 117. A description of a low adhesion backing material suitable for use with medical dressings of the present disclosure can be found in U.S. Pat. Nos. 5,531,855 and 6,264,976, which are incorporated herein by reference in their entirety.

Suitable carrier materials can include, but are not limited to, polyethylene/vinyl acetate copolymer-coated papers and polyester films. One example of a suitable carrier material is a polyethylene/vinyl acetate copolymer coated super calendared Kraft paper (1-80BKG-157 PE; LOPAREX of Willowbrook, Ill.).

In such embodiments, the base dressing 104B can include an inner, or center, portion 123 on which a bracket 102B can be mounted, and an outer (e.g., annular) window portion 127 that can be substantially transparent, but this need not be the case. It should be understood that the term “annular” is not limited only to circular shapes, but can refer to a structure having an outer edge and an inner edge, such that the inner edge defines an opening. Such a structure can have any shape necessary, including, but not limited to, circular, triangular, rectangular, square, trapezoidal, polygonal, etc., or combinations thereof. The window portion 127 can allow a user to see underneath the base dressing 104B (e.g., to the underlying skin or tissue) while the base dressing 104B remains coupled to the subject.

As a result of the features shown in FIGS. 4 and 4A, it is clear how a graphic 108 can be employed on various disposable portions of a medical article securement system of the present disclosure. In addition, in some embodiments, a disposable portion of a system of the present disclosure can include a graphic of the present disclosure having a shape, or being shaped into, at least one feature of a graphic of the present disclosure. For example, in some embodiments, a disposable portion of the system (e.g., a release liner, a carrier, a frame, etc.) can be shaped into that of an anatomical feature (i.e., a first feature of a graphic) and/or a non-anatomical feature, such as a directional cue (i.e., a second feature of a graphic).

In the embodiments shown in FIGS. 1-4A, the graphic 108 includes a first feature 106 and a second feature 107, as described above. Particularly, the second feature 107 is used to provide additional information and is positioned relative to the first feature 106 to provide additional guidance for coupling the system 100 to the subject S in a desired orientation and/or for coupling the medical article 60 in a desired orientation. By way of example only, the first feature 106 and the second feature 107 of the graphics 108 are located adjacent one another. That is, the first feature 106 and the second feature 107 are located side-by-side, with the first feature 106 on the left and the second feature 107 on the right. However, other variations of relative positioning are possible. In some embodiments, the second feature 107 can be positioned on the left side of the first feature 106. In some embodiments, the first feature 106 can be located above or below the second feature 107. In some embodiments, the first feature 106 can include an outline (i.e., with a white fill, or no fill, inside the outline) of an anatomical feature, and the second feature 107 can be located within the outline of the first feature 106. In some embodiments, the second feature 107 can include an outline (i.e., with white fill, or no fill, inside the outline), and the first feature 106 can be located within the outline of the second feature 107.

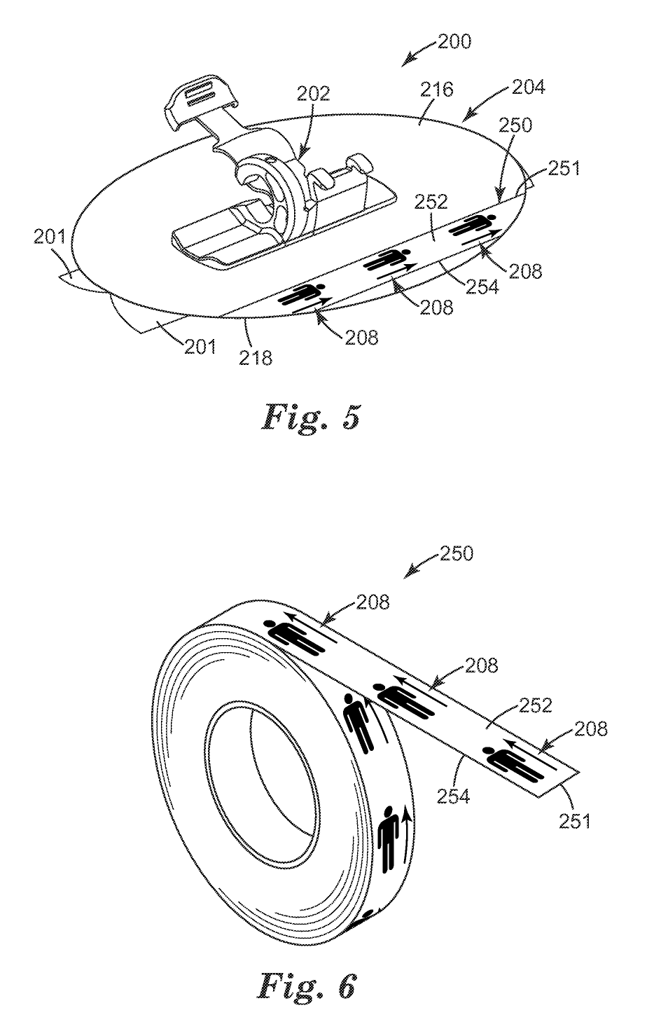

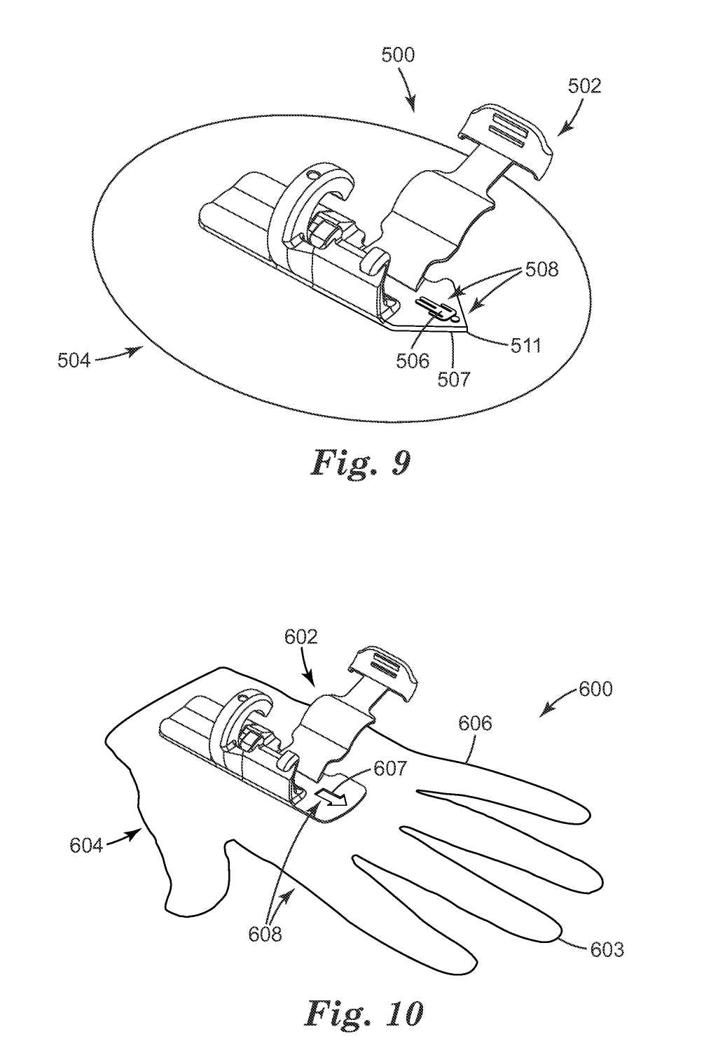

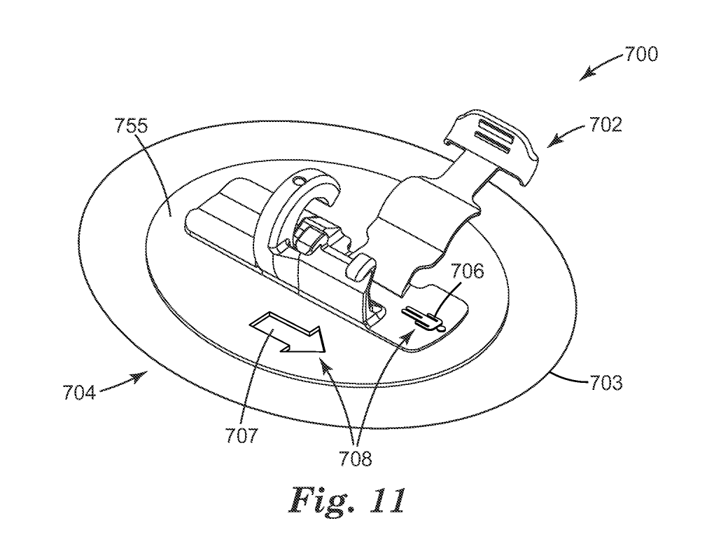

Furthermore, the graphic 108 is shown as being printed on the system 100, and particularly, as being printed on the base dressing 104, or a disposable portion of the system 100 (e.g., a release liner 101 or a carrier 117). However, the graphic 108 can be provided by or coupled to the system 100 in other ways. For example, the graphics of the present disclosure can be integrally formed with a portion of the system, such as by molding, embossing, embedding (e.g., into a transparent portion of the system), etching, other suitable processing means, or a combination thereof (see, e.g., FIGS. 8 and 9). Alternatively, or additionally, as described in greater detail below, at least a portion of the graphic can be provided by an article (e.g., a tape, a sticker on a release surface, etc.) that is coupled to (e.g., adhered to) at least a portion of the system (see FIGS. 5 and 6); at least a portion of the graphic can be provided by at least a portion of the system being shaped to form the graphic (see FIGS. 8-10); at least a portion of the graphic can be provided by a cutout, or by material being removed from a portion of the system (see FIGS. 10 and 11); or a combination thereof. That is, in some embodiments, the graphic can be formed by additive and/or subtractive methods.

Examples of “printing” a graphic of the present disclosure can include, but are not limited to, applying a print material (e.g., an ink, a toner, and/or a polymeric material) in the form of writing, printing (e.g., inkjet, laser, etc.) and/or staining; laser marking (e.g., employing light-sensitive pigments, as described in U.S. Patent Publication No. 2013/0337213, “Laser Marking Process and Articles,” which is incorporated herein by reference); other suitable printing techniques; and combinations thereof.

In some embodiments, in order for the graphic 108 to be easily identifiable by a user, the graphic 108 can have an optical property that is different from at least an adjacent portion of the system 100. Such optical properties can include, but are not limited to, color or shade, transparency/translucency/opacity, reflectance, gloss or shine, refractive index, other suitable optical properties, or combinations thereof. Such optical properties can typically be visually distinguishable by the naked human eye.

In use, a user can use the graphic 108 as a visual cue to quickly and easily determine how to orient and couple the system 100 to the subject S, which can ultimately aid in orienting and coupling the medical article 60 (or any medical article of interest) in a proper orientation relative to the system 100 and/or the subject S. That is, in some embodiments, coupling the medical article 60 to the system 100 and/or coupling the system 100 to the subject S can be done according to, or as instructed by, the graphic 108. In some embodiments, the medical article can be coupled to the system 100 prior to coupling the system 100 to the subject S, and in some embodiments, the medical article can be coupled to the system 100 after coupling the system 100 to the subject S.

As mentioned above, in some embodiments, coupling the system 100 to the subject S can include adhering at least a portion of the system 100 (e.g., the base dressing 104) to the subject S. In some embodiments, coupling the system 100 to the subject S can include mechanically coupling at last a portion of the system 100 to the subject S, as exemplified in FIG. 1A, e.g., by wrapping the system 100 about a portion of the subject S (e.g., a limb) and/or securing the system 100 with any variety of fasteners.

Furthermore, in some embodiments, at least a portion of a medical article can be adhered to the system 100, e.g., via one or more adhesive layers on the medical article, the system 100, or both. In some embodiments, at least a portion of a medical article can be mechanically coupled to the system 100, e.g., via the bracket 102.

Additional exemplary embodiments of graphics and medical article securement systems of the present disclosure will now be described with respect to FIGS. 5-11. FIGS. 5-11 illustrate various graphics and systems of the present disclosure, wherein like numerals represent like elements. The graphics of FIGS. 5-11 share many of the same elements, features, and functions as the system 100 and graphic 108 described above with respect to FIGS. 1-4A. Reference is made to the description above accompanying FIGS. 1-4A for a more complete description of the features and elements (and alternatives to such features and elements) of the embodiments illustrated in FIGS. 5-11. Any of the features described above with respect to FIGS. 1-4A can be applied to the embodiments of FIGS. 5-11, and vice versa. The same medical article 60 illustrated in FIG. 1 as being coupled to the bracket 102 can also be used with each of the systems of FIGS. 5-11.

FIG. 5 illustrates a medical article securement system 200 according to another embodiment of the present disclosure. The system 200 includes a bracket 202; a base dressing 204 having a first surface 216 and a second surface 218; two release liners 201 in a butterfly configuration; and an article 250 (comprising a graphic 208) that is coupled to the first surface 216 of the base dressing 204. By way of example only, the article 250 is illustrated as including a plurality of graphics 208 in a repeating fashion. The article 250 can be referred to as being coupled to the system 200, or as forming a portion of the system 200.

The article 250 is illustrated as a tape or sticker, which can include a backing 251 having a first side or surface 252 on which the graphics 208 are provided, and a second side or surface 254 that is coupled (e.g., via an adhesive) to the first surface 216 of the base dressing 204.

Suitable backings can include, but are not limited to, films (e.g., formed of vinyl, polyurethane, polyester, polyethylene, or combinations thereof); wovens or non-wovens (e.g., formed of polyester); hook-and-loop materials or articles; or combinations thereof.

In some embodiments, backings for articles of the present disclosure comprising the graphic can be thin, flexible and breathable.

FIG. 6 illustrates the article 250 prior to being coupled to the system 200. Particularly, FIG. 6 illustrates the article 250 in the form of a roll of tape comprising the graphics 208, such that the article 250 is in a rolled form or configuration. Other embodiments of the article 250 can include a stack or pad configuration. The second side 254 can include an adhesive, including, but not limited to, a pressure-sensitive adhesive, a heat-seal adhesive, a cold-seal adhesive, another suitable type of adhesive, or a combination thereof. The adhesive on the second side 254 can be different from the skin-contact adhesive 115, and in some embodiments, enhanced adhesion to the system 200 and/or any medical articles coupled thereto can be enhanced by employing an adhesive that is different from the skin-contact adhesive 115.

In the roll or stack, the adhesive on the second side 254 can be in contact with the first side 252 of the backing 251 of an underlying sheet or portion of the article 250. In such embodiments, the first side 252 can include a release coating (e.g., low adhesion backsize (LAB) coating) to allow the resulting tape to unwind from itself when wound in a roll or to release when in a pad form. When utilized, the release coating composition should be compatible with the adhesive composition and not degrade the adhesive properties of the tape, such as by being transferred to the adhesive composition. Alternatively, as is generally understood in the art, the roll can include a release liner over the pressure-sensitive adhesive on the second side 254. In some embodiments, the roll can include a release liner onto which a plurality of stickers can be applied, and each sticker can include one or more of the graphics 208.

In embodiments in which the roll or stack comprising the article 250 includes a release liner, it should be understood that the term “article” as described herein as comprising a graphic of the present disclosure generally refers to the article after any disposable portions (e.g., release liners) have been removed to allow the portion comprising the graphic to be coupled to the another portion of the system.

Suitable pressure-sensitive adhesives for use with the article 250 can include, but are not limited to, acrylates, silicones, polyisobutylenes, synthetic rubbers, natural rubbers, and combinations thereof. Suitable heat-seal adhesives can include, but are not limited to, polyurethanes, polyamides, polyesters, polyolefins, other suitable heat-seal adhesives, or a combination thereof. Suitable cold-seal adhesives can include, but are not limited to, natural rubber, polyisoprene, butyl rubber, silicones, and combinations thereof.

In some embodiments, the article 250 can be coupled to the system 200 via an electrostatic charge (e.g., as employed in a window sticker). In some embodiments, the article 250 can include an electrostatic charge that is imparted to the backing 251. Alternatively, in some embodiments, the article 250 can include a combination of an electrostatic charge with other adhesive components, such as any of those described above. Articles 250 including an electrostatic charge can have a permanent electrostatic charge and can exhibit electrostatic attraction to a wide variety of surfaces thereby allowing the backing 251 to be removably adhered to the desired surface. In some embodiments, permanent electrostatic charge can be imparted to the article 250 using corona charging (e.g., nitrogen or air).

Suitable release coating compositions for the LAB layer of tapes can include silicone, alkyl, or fluorochemical constituents, or combinations thereof, as the release imparting component. Examples of useful release coating compositions can include silicone containing polymers, such as silicone polyurethanes, silicone polyureas and silicone polyurethane/ureas; and silicone acrylate grafted copolymers. Other useful release coating compositions can include fluorochemical containing polymers, and polymers containing long alkyl side chains such as polyvinyl N-alkyl carbamates (e.g., polyvinyl N-octadecyl carbamates), and copolymers containing higher alkyl acrylates (e.g., octadecyl acrylate or behenyl acrylate) or alkyl methacrylates (e.g., stearyl methacrylate), where the alkyl side chain includes from about 16 to 22 carbon atoms.

By way of example only, each graphic 208 on the article 250 is substantially the same as the graphic 108 of FIGS. 1-4A. The graphics 208 are shown as being formed on the first side 252 of the backing 252, e.g., by printing. However, the graphic 208 can additionally or alternatively be formed on or with the article 250 by molding, embossing, embedding, etching, other suitable processing means, or a combination thereof.

Furthermore, while the graphic 208 is illustrated as being provided on the first side 252 of the article 250 (i.e., the backing 251), it should be understood that the graphic 208 can additionally or alternatively be provided (i.e., by any of the above-described means) on the second side 254 of the article 250, e.g., if the backing is substantially transparent. In still other embodiments, the graphic 208 can be additionally or alternatively be provided in between the first side 252 and the second side 254 (e.g., by being embedded within, or between layers of, the tape).

While an adhesive tape is illustrated in FIGS. 5 and 6 by way of example, it should be understood that the article 250 can be configured to be coupled to any portion of the system 200 by any of a variety of coupling means. Such coupling means can include, but are not limited to, stitches, staples, screws, nails, rivets, brads, crimps, welding (e.g., sonic (e.g., ultrasonic) welding), any thermal bonding technique (e.g., heat and/or pressure applied to one or both of the components to be coupled), snap-fit engagement, press-fit engagement, heat sealing, hook-and-loop fasteners, other suitable permanent or semi-permanent coupling means, and combinations thereof.

In addition, while the article 250 is shown in FIG. 5 as being coupled to the base dressing 104, it should be understood that the article 250 can additionally or alternatively be coupled to another portion of the system 200, such as the bracket 202, or a disposable portion, such as a release liner 201.

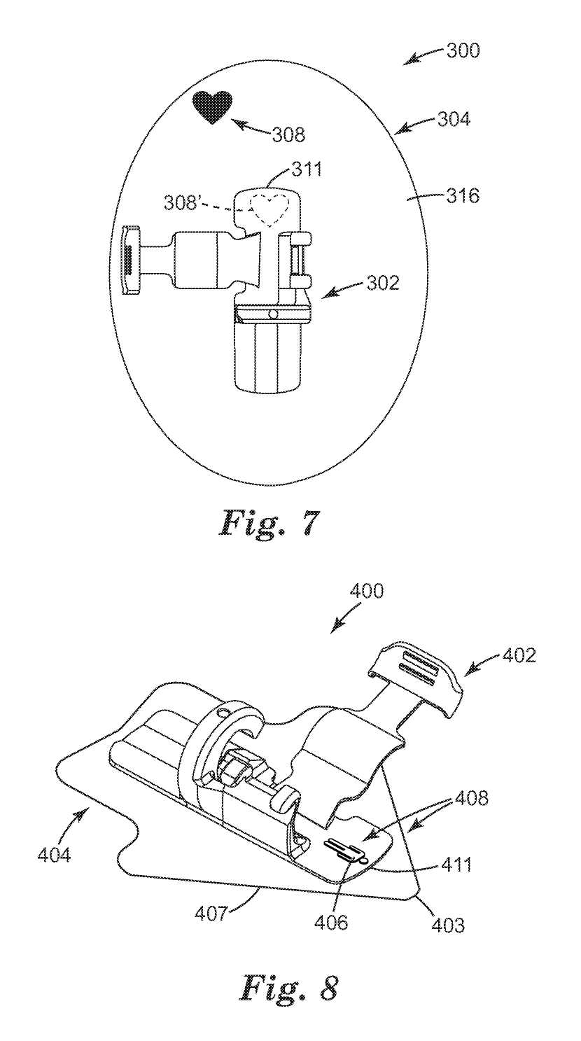

FIG. 7 illustrates a medical article securement system 300 according to another embodiment of the present disclosure. The system 300 includes a bracket 302; a base dressing 304; and a graphic 308. The graphic 308 includes only a representation of an anatomical feature, i.e., the graphic 308 does not include a second, non-anatomical feature. In addition, by way of example, the graphic 308 includes a simple or cartoon-like representation of a heart. The graphic 308 and system 300 of FIG. 7 illustrate that the graphics of the present disclosure need not be anatomically accurate to provide sufficient and clear information to a user.

The graphic 308 is located toward or adjacent one end of the base dressing 304, i.e., a distal end 303 of the base dressing 304, to indicate that the distal end 303 of the base dressing 304 should be oriented to point toward the heart of the subject to which the system 300 is to be coupled. The graphic 308 is further positioned more toward one lateral side of the system 300 (e.g., the base dressing 304), which can indicate to a user that the heart should be above and to the left of the system 300 when the system is coupled to the subject. Other positioning cues are possible, depending on the specific location of the graphic 308.

While the graphic 308 is shown as being located on the base dressing 304, it should be understood that the graphic 308 can additionally or alternatively be located on another portion of the system 300, such as the bracket 302, or a disposable portion, such as a release liner. As shown with a dashed line in FIG. 7, a second graphic 308′ (which is shown by way of example as being the same as the graphic 308) can be employed in addition to, or instead of, the graphic 308, which is shown as being provided on the bracket 302. Particularly, the second graphic 308′ is shown as being located toward a distal, longitudinal end 311 of the bracket 302 to indicate that the distal end 311 of the bracket 302 should be oriented toward the heart.

Furthermore, while the graphic 308 is shown as being provided on a first surface 316 of the base dressing 304, it should be understood that the graphic 308 can additionally or alternatively be provided on a second (or back) surface (not shown) of the base dressing 304, as shown in FIG. 4 and described above.

FIG. 8 illustrates a medical article securement system 400 according to another embodiment of the present disclosure. The system 400 includes a bracket 402; a base dressing 404; and a graphic 408.

The graphic 408 includes a first feature 406 in the form of an outline of a human body on the bracket 402. Particularly, the first feature 406 of the graphic 408 is embossed on, or molded into, the bracket 402; however, any of the above-described means for forming or providing the graphic 408 can additionally or alternatively be employed.

The graphic 408 further includes a second feature 407 in the form of a directional cue, namely, an arrow. Particularly, the base dressing 404 is itself shaped into an arrow, and the point of the arrow is at a distal end 403 of the base dressing 404. The base dressing 404 is therefore an example of how at least a portion of the system 400 can have a shape, outline or periphery that defines at least a portion of the graphic 408.

The combination of the first feature 406 and the second feature 407 indicates to a user that the distal end 403 of the base dressing 404, a distal end 411 of the bracket 402 (and a distal end of the system 400 as a whole) should be oriented toward the head of the subject. While the base dressing 404 is shown as having its entirety shaped into the second feature 407, it should be understood that in some embodiments, only a portion of the base dressing 404 (e.g., a cushioning foam layer, as described below and illustrated in FIG. 11) is shaped into the second feature 407.

The graphic 408 is an example of the first feature 406 and the second feature 407 being separate from one another and even being provided on or by different portions of the system 400. By way of example, the first feature 406 of the graphic 408 itself is located toward a distal longitudinal end 411 of the bracket 402 to indicate that the distal end 411 of the bracket 402 (or of the system 400 as a whole) should be oriented toward the head of the subject. As a result, the second feature 407 may not be necessary, but may provide an additional visual cue to a user to further facilitate proper orientation of a medical article and/or the system 400 relative to a subject.

As described with respect to embodiments above, the system 400 can further include additional or alternative graphics that are provided on other portions of the system 400, such as disposable portions (not shown). Furthermore, the first feature 406 is shown as being provided on the bracket 402, but it should be understood that the first feature 406 can instead be provided on or by the base dressing 404 and/or another portion of the system 400, and the specific arrangement of the graphic 408 is shown in FIG. 7 by way of example only.

FIG. 9 illustrates a medical article securement system 500 according to another embodiment of the present disclosure. The system 500 includes a bracket 502; a base dressing 504; and a graphic 508 comprising a first feature 506 and a second feature 507. The system 500 is similar to the system 400 of FIG. 8, except that the second feature 507 (i.e., the arrow) is provided by the bracket 502, and particularly by a distal end 511 of the bracket 502, instead of the base dressing 504. Rather, the base dressing 504 has an oval shape. It should be understood that the shapes of the base dressings described and illustrated herein that do not form a portion of the graphic can be any standard shape desired, such as oval, round, rectangular, square, triangular, another suitable shape, or a combination thereof.

The distal end 511 of the bracket 502 is shaped into a point or an arrow. As a result, the combination of the first feature 506 and the second feature 507 of the graphic 508 indicates to a user that the distal end 511 of the bracket 502 (or a distal end of the system 500 as a whole) should be oriented toward the head of the subject. It should be understood, however, that the opposite configuration can also be employed, where the bracket 502 can instead be shaped into a first feature 506 of the graphic 508 (i.e., a representation of an anatomical feature), and the second feature 507 (i.e., the non-anatomical feature), if employed, can be provided on the bracket 502 or on another portion of the system 500. All other features and alternatives thereto described with respect to the earlier embodiments can also be applied to the system 500 of FIG. 9. For example, other means of forming the first feature 506 and/or the second feature 507 can be employed, and the system 500 can additionally or alternatively include graphics 508 (or features thereof) located on other portions of the system 500.

FIG. 10 illustrates a medical article securement system 600 according to another embodiment of the present disclosure. The system 600 includes a bracket 602; a base dressing 604; and a graphic 608 comprising a first feature 606 and a second feature 607. The system 600 is similar to the system 400 of FIG. 8, except that the first feature 606 is provided by the base dressing 604 (i.e., by the base dressing 604 being shaped into a representation of an anatomical feature, namely, a hand), and the second feature 607 is provided on the bracket 602. While the base dressing 604 is shown as having its entirety shaped into the first feature 606, it should be understood that in some embodiments, only a portion of the base dressing 604 (e.g., a cushioning foam layer, as described below and illustrated in FIG. 11) is shaped into the first feature 606.

The second feature 607 includes an arrow, which is pointing toward a distal end 611 of the bracket 602 and toward the fingers of the hand of the first feature 606, i.e., toward a distal end 603 of the base dressing 604. As a result, the combination of the first feature 606 and the second feature 607 indicates to a user that the distal end 611 of the bracket 602 and the distal end 603 of the base dressing 604 (or a distal end of the system 600 as a whole) should be oriented toward the fingers of a subject's hand.

The second feature 607 is shown by way of example only as being a cutout from the bracket 602. That is, the shape of the arrow is formed in the bracket 602 by molding (i.e., is integrally formed) or by removing material from the bracket 602. The cutout can include a partial (e.g., a recess) or full cutout from the bracket 602. That is, in some embodiments, the second feature 607 can form an aperture through the entire thickness of the bracket 602, but that need not be the case.

All other features and alternatives thereto described with respect to the earlier embodiments can also be applied to the system 600 of FIG. 10. For example, other means of forming the first feature 606 and/or the second feature 607 can be employed, and the system 600 can additionally or alternatively include graphics 608 (or features thereof) located on other portions of the system 600.

FIG. 11 illustrates a medical article securement system 700 according to another embodiment of the present disclosure. The system 700 includes a bracket 702; a base dressing 704; and a graphic 708 comprising a first feature 706 and a second feature 707.

As shown in FIG. 11, in some embodiments, the base dressing 704 can include a foam 755 (e.g., a cushioning foam layer) or other wound treatment or drug delivery vehicle. In some embodiments, one or more other dressing layers can cover the foam 755, can be located under the foam 755, or both (i.e., such that the foam 755 is sandwiched between dressing layers).

The system 700 is similar to the system 400 of FIG. 8 and the system 500 of FIG. 9, except that the second feature 707 is not provided by forming an element of the system 700 into a desired non-anatomical shape. Rather, the second feature 707 is provided by a cutout from the base dressing 704, and particularly, by a cutout from the foam 755 of the base dressing 704. The second feature 707 is in the form of a directional cue, and particularly, an arrow, and like the previous embodiments, the arrow points in the same direction as the head of the figure outline of the first feature 706, indicating to a user that the system 700 should be oriented such that a distal end thereof (e.g., a distal end 703 of the base dressing 704) should be oriented toward the head of the subject.

The cutout second feature 707 is shown by way of example only, and it should be understood that the base dressing 704 need not include the foam 755, and the foam 755 need not be the portion of the base dressing 704 that provides the second feature 707. Furthermore, while a portion of the graphic 708 (i.e., the second feature 707) is shown as being a cutout from a portion of the base dressing 704, in some embodiments, a portion of the graphic 708 can additionally or alternatively be a cutout (partial or full) from the bracket 702. All other features and alternatives thereto described with respect to the earlier embodiments can also be applied to the system 700 of FIG. 11. For example, other means of forming the first feature 706 and/or the second feature 707 can be employed, and the system 700 can additionally or alternatively include graphics 708 (or features thereof) located on other portions of the system 700.

Each embodiment shown in the figures is illustrated as a separate embodiment for clarity in illustrating a variety of features of the graphics and medical article securement systems of the present disclosure. However, it should be understood that any combination of elements and features of any of the embodiments illustrated in the figures and described herein can be employed in the graphics and medical article securement system of the present disclosure.

The following embodiments are intended to be illustrative of the present disclosure and not limiting.

### EMBODIMENTS

1. A graphic for use with a medical article securement system, the graphic comprising a representation of an anatomical feature, such that the graphic provides a visual cue for coupling the medical article securement system to a subject in a desired orientation relative to the subject.

2. The graphic of embodiment 1, wherein the graphic is provided on a roll.

3. An article comprising the graphic of embodiment 1 or 2.

4. The article of embodiment 3, further comprising a backing having a first side and a second side opposite the first side; and wherein the second side of the backing is configured to be coupled to the medical article securement system.

5. The article of embodiment 4, wherein the graphic is provided on the first side of the backing.

6. The article of embodiment 4 or 5, wherein the graphic is provided on the second side of the backing.

7. The article of any of embodiments 4-6, wherein the second side of the backing is configured to be coupled to the medical article securement system.

8. The article of any of embodiments 3-7, wherein the article is provided in the form of a roll.

9. The article of any of embodiments 3-8, wherein the article includes a sticker.

10. The article of any of embodiments 3-9, wherein the article includes a tape.

11. A medical article securement system, the system comprising:
a graphic comprising a representation of an anatomical feature, such that the graphic provides a visual cue for coupling the medical article securement system to a subject in a desired orientation relative to the subject;
the system configured to be coupled to the subject and further configured to secure at least a portion of a medical article.

12. A method for coupling a medical article securement system to a subject, the method comprising:
providing a medical article securement system comprising a graphic, the graphic comprising a representation of an anatomical feature, such that the graphic provides a visual cue for coupling the medical article securement system to a subject in a desired orientation relative to the subject; and
coupling the medical article securement system to the subject according to the graphic.

13. The system of embodiment 11 or the method of embodiment 12, wherein the graphic is integrally formed with the medical article securement system.

14. The system of embodiment 11 or 13 or the method of embodiment 12 or 12, wherein the graphic is printed on the medical article securement system.

15. The system of any of embodiments 11 and 13-14 or the method of any of embodiments 12-14, wherein the medical article securement system has a first end and a second end, and wherein the graphic is located toward the first end to indicate that the first end of the medical article securement system is to be oriented toward the anatomical feature of the subject that is represented by the graphic.

16. The system of any of embodiments 11 and 13-15 or the method of any of embodiments 12-15, wherein at least a portion of the medical article securement system is shaped to provide at least a portion of the graphic.

17. The system or method of embodiment 16, wherein at least a portion of the medical article securement system is shaped into the representation of an anatomical feature.

18. The system of any of embodiments 11 and 13-17 or the method of any of embodiments 12-17, wherein the representation of an anatomical feature is a first feature of the graphic, and further comprising a second feature that is non-anatomical, and wherein at least a portion of the medical article securement system is shaped into at least one of the first feature and the second feature.

19. The system of any of embodiments 11 and 13-18 or the method of any of embodiments 12-18, wherein the medical article securement system includes a bracket configured to secure a medical article.

20. The system or method of embodiment 19, wherein the bracket includes at least a portion of the graphic.

21. The system or method of embodiment 19 or 20, wherein the bracket is shaped into at least a portion of the graphic.

22. The system of any of embodiments 11 and 13-21 or the method of any of embodiments 12-21, wherein the medical article securement system includes a disposable portion, wherein the disposable portion includes a liner, a carrier, a frame, or a combination thereof, and wherein the disposable portion includes at least a portion of the graphic.

23. The system of any of embodiments 11 and 13-22 or the method of any of embodiments 12-22, wherein the medical article securement system includes a base dressing configured to be coupled to the subject, and wherein the base dressing includes at least a portion of the graphic.

24. The system or method of embodiment 23, wherein the base dressing is shaped to provide at least a portion of the graphic.

25. The system or method of embodiment 23 or 24, wherein the base dressing is shaped into the representation of an anatomical feature.

26. The system or method of any of embodiments 23-25, wherein the representation of an anatomical feature is a first feature of the graphic, and wherein the base dressing is shaped into a second feature of the graphic that is non-anatomical.

27. The system or method of embodiment 26, wherein the second feature includes a directional cue.

28. The system or method of embodiment 26 or 27, wherein the directional cue includes an arrow.

29. The system of any of embodiments 11 and 13-28 or the method of any of embodiments 12-28, wherein the graphic has an optical property that is different from at least an adjacent portion of the medical article securement system.

30. The system or method of embodiment 29, wherein the optical property includes at least one of color, transparency, reflectance, gloss, and a combination thereof.

31. The system of any of embodiments 11 and 13-30 or the method of any of embodiments 12-30, wherein the graphic includes a cutout from a portion of the medical article securement system.

32. A medical article securement system, the system comprising:
a base dressing configured to be coupled to a subject;
a bracket coupled to the base dressing, the bracket configured to secure at least a portion of a medical article; and
a graphic comprising a representation of an anatomical feature.

33. A method for coupling a medical article securement system to a subject, the method comprising:
providing a medical article securement system comprising:
   a base dressing configured to be coupled to a subject,
   a bracket coupled to the base dressing, the bracket configured to secure at least a portion of a medical article, and
   a graphic comprising a representation of an anatomical feature; and
coupling the medical article securement system to the subject according to the graphic.

34. The method of any of embodiments 12-31 and 33, further comprising coupling a medical article to the medical article securement system.

35. The method of embodiment 34, wherein coupling the medical article to the medical article securement system occurs prior to coupling the medical article securement system to a subject.

36. The method of embodiment 34, wherein coupling the medical article securement system to a subject occurs prior to coupling the medical article to the medical article securement system.

37. The method of any of embodiments 34-36, wherein coupling a medical article to the medical article securement system includes adhering the medical article to the medical article securement system.

38. The method of any of embodiments 34-37, wherein coupling a medical article to the medical article securement system includes mechanically coupling the medical article to the medical article securement system.

39. The method of any of embodiments 12-31 and 33-38, further comprising orienting the medical article securement system with respect to the subject according to the graphic prior to coupling the medical article securement system to the subject.

40. The method of any of embodiments 12-31 and 33-39, wherein coupling the medical article securement system to the subject includes adhering at least a portion of the medical article securement system to the subject.

41. The method of any of embodiments 12-31 and 33-40, wherein coupling the medical article securement system to the subject includes mechanically coupling at least a portion of the medical article securement system to the subject.

42. The system of embodiment 32 or the method of any of embodiments 33-41, wherein the bracket is rigid relative to the base dressing.

43. The system of embodiment 32 or 42 or the method of any of embodiments 33-42, wherein the system is configured to be coupled to the subject and is further configured to secure at least a portion of a medical article to inhibit movement of the medical article in at least one of a longitudinal direction, a lateral direction, and a normal direction.

44. The system of any of embodiments 32 and 42-43 or the method of any of embodiments 33-43, wherein the graphic provides a visual cue for coupling the medical article securement system to a subject in a desired orientation relative to the subject.

45. The system of any of embodiments 32 and 42-44, further comprising:
a first side provided by at least one of the base dressing and the bracket and positioned to face away from the subject when the medical article securement system is coupled to the subject; and
a second side opposite the first side.

46. The system of embodiment 45, wherein the bracket is configured to inhibit movement of the medical article in at least one of a longitudinal direction, a lateral direction, and a normal direction relative to the first side.

47. The system of embodiment 45 or 46, wherein the graphic is visible from at least one of the first side and the second side.

48. The system of any of embodiments 32 and 42-47, further comprising an article coupled to another portion of the system, the article comprising the graphic.

49. The system of embodiment 48, wherein the article includes a backing having a first side and a second side; and wherein the second side is coupled to another portion of the system.

50. The system of embodiment 49, wherein the graphic is provided on the first side of the article.

51. The system of embodiment 49 or 50, wherein the graphic is provided on the second side of the article.

52. The system of any of embodiments 49-51, wherein the backing includes an adhesive on its second side.

53. The system of any of embodiments 48-52, wherein the article is a tape or a sticker.

54. The system of any of embodiments 48-53, wherein the article is coupled to at least one of the base dressing, the bracket, and a disposable portion of the medical article securement system.

55. The system of any of embodiments 48-54, wherein the article is coupled to at least one of the base dressing, the bracket, a liner, a carrier, and a frame.

56. The system of any of embodiments 32 and 42-55 or the method of any of embodiments 33-44, wherein the graphic is provided by the base dressing.

57. The system of any of embodiments 32 and 42-56 or the method of any of embodiments 33-44 and 56, wherein the base dressing includes a first surface and a second surface opposite the first surface, wherein the second surface is configured to be coupled to the subject, and wherein the graphic is provided on the first surface of the base dressing.

58. The system of any of embodiments 32 and 42-57 or the method of any of embodiments 33-44 and 56-57, wherein the graphic is provided by the bracket.

59. The system of any of embodiments 32 and 42-58 or the method of any of embodiments 33-44 and 56-58, wherein the bracket includes a first surface and a second surface opposite the first surface, wherein the second surface is coupled to the base dressing.

60. The system or method of embodiment 59, wherein the graphic is provided on the first surface of the bracket.

61. The system or method of embodiment 59 or 60, wherein the graphic is provided on the second surface of the bracket.

62. The system of any of embodiments 32 and 42-61 or the method of any of embodiments 33-44 and 56-61, wherein the base dressing is shaped to provide at least a portion of the graphic.

63. The system of any of embodiments 32 and 42-62 or the method of any of embodiments 33-44 and 56-62, wherein the medical article includes a catheter.

64. The system of any of embodiments 32 and 42-63 or the method of any of embodiments 33-44 and 56-63, wherein the bracket is configured to retain at least a portion of the catheter to inhibit movement of the retained portion of the catheter in at least one of a longitudinal direction, a lateral direction, and a normal direction, relative to the bracket.

65. The system of any of embodiments 32 and 42-64 or the method of any of embodiments 33-44 and 56-64, wherein the graphic is integrally formed with at least one of the base dressing and the bracket.

66. The system of any of embodiments 32 and 42-65 or the method of any of embodiments 33-44 and 56-65, wherein the graphic is printed on at least one of the base dressing, the bracket, and a disposable portion of the medical article securement system.

67. The system of any of embodiments 32 and 42-66 or the method of any of embodiments 33-44 and 56-66, wherein the graphic is printed on at least one of the base dressing, the bracket, a liner, a carrier, and a frame.

68. The system of any of embodiments 32 and 42-67 or the method of any of embodiments 33-44 and 56-67, wherein the medical article securement system has a first end and a second end, and wherein the graphic is located toward the first end of the medical article securement system to indicate that the first end of the medical article securement system is to be oriented toward the anatomical feature of the subject that is represented by the graphic.

69. The system of any of embodiments 32 and 42-68 or the method of any of embodiments 33-44 and 56-68, wherein the base dressing includes a first end and a second end, and wherein the graphic is located toward the first end of the base dressing to indicate that the first end of the base dressing is to be oriented toward the anatomical feature of the subject that is represented by the graphic.

70. The system of any of embodiments 32 and 42-69 or the method of any of embodiments 33-44 and 56-69, wherein the bracket has a first end and a second end, and wherein the graphic is located toward the first end of the bracket to indicate that the first end of the bracket is to be oriented toward the anatomical feature of the subject that is represented by the graphic.

71. The system of any of embodiments 32 and 42-70 or the method of any of embodiments 33-44 and 56-70, wherein at least a portion of the medical article securement system is shaped to provide at least a portion of the graphic.

72. The system or method of embodiment 71, wherein at least a portion of the medical article securement system is shaped into the representation of an anatomical feature.

73. The system of any of embodiments 32 and 42-72 or the method of any of embodiments 33-44 and 56-72, wherein at least a portion of the base dressing is shaped to provide at least a portion of the graphic.

74. The system or method of embodiment 73, wherein at least a portion of the base dressing is shaped into the representation of an anatomical feature.

75. The system or method of embodiment 73 or 74, wherein the representation of an anatomical feature is a first feature of the graphic, and wherein the base dressing is shaped into a second feature of the graphic that is non-anatomical.

76. The system or method of embodiment 75, wherein the second feature includes a directional cue.

77. The system or method of embodiment 76, wherein the directional cue includes an arrow.

78. The system of any of embodiments 32 and 42-77 or the method of any of embodiments 33-44 and 56-77, wherein at least a portion of the bracket is shaped to provide at least a portion of the graphic.

79. The system or method of embodiment 78, wherein at least a portion of the bracket is shaped into the representation of an anatomical feature.

80. The system or method of embodiment 78 or 79, wherein the representation of an anatomical feature is a first feature of the graphic, and wherein the bracket is shaped into a second feature of the graphic that is non-anatomical.

81. The system or method of embodiment 80, wherein the second feature includes a directional cue.

82. The system or method of embodiment 81, wherein the directional cue includes an arrow.

83. The system of any of embodiments 32 and 42-82 or the method of any of embodiments 33-44 and 56-82, wherein the representation of an anatomical feature is a first feature of the graphic, and further comprising a second feature that is non-anatomical, and wherein at least one of the base dressing and the bracket is shaped into at least one of the first feature and the second feature.

84. The system of any of embodiments 32 and 42-83 or the method of any of embodiments 33-44 and 56-83, wherein the graphic has an optical property that is different from at least an adjacent portion of the medical article securement system.

85. The system or method of embodiment 84, wherein the optical property includes at least one of color, transparency, reflectance, gloss, and a combination thereof.

86. The system of any of embodiments 32 and 42-85 or the method of any of embodiments 33-44 and 56-85, wherein the graphic includes a cutout from at least one of the base dressing and the bracket.

87. The graphic of embodiment 1 or 2, the article of any of embodiments 3-10, the system of any of embodiments 11, 13-32 and 42-86, or the method of any of embodiments 12-31, 33-44 and 56-86, wherein the anatomical feature includes at least one of a whole body, an upper body, a lower body, and a body part.

88. The graphic of any of embodiments 1, 2 and 87, the article of any of embodiments 3-10 and 87, the system of any of embodiments 11, 13-32 and 42-87, or the method of any of embodiments 12-31, 33-44 and 56-87, wherein the anatomical feature includes at least one of a body part that is visible on the subject or a body part that is not visible on the subject.

89. The graphic of any of embodiments 1, 2 and 87-88, the article of any of embodiments 3-10 and 87-88, the system of any of embodiments 11, 13-32 and 42-88, or the method of any of embodiments 12-31, 33-44 and 56-88, wherein the anatomical feature includes at least a portion of at least one of a limb, a hand, a foot, a finger, a toe, a head, a neck, a torso, an internal organ, and a combination thereof.

90. The graphic of any of embodiments 1, 2 and 87-89, the article of any of embodiments 3-10 and 87-89, the system of any of embodiments 11, 13-32 and 42-89, or the method of any of embodiments 12-31, 33-44 and 56-89, wherein the anatomical feature includes at least a portion of at least one of a heart, a stomach, a bladder, a brain, a lung, a skeletal feature, and a combination thereof.

91. The graphic of any of embodiments 1, 2 and 87-90, the article of any of embodiments 3-10 and 87-90, the system of any of embodiments 11, 13-32 and 42-90, or the method of any of embodiments 12-31, 33-44 and 56-90, wherein the representation of an anatomical feature includes at least one of an outline, a cartoon, an anatomically-accurate graphic, and a combination thereof.

92. The graphic of any of embodiments 1, 2 and 87-91, the article of any of embodiments 3-10 and 87-91, the system of any of embodiments 11, 13-32 and 42-91, or the method of any of embodiments 12-31, 33-44 and 56-91, wherein the graphic provides a visual cue for coupling a medical article to the medical article securement system.

93. The graphic of any of embodiments 1, 2 and 87-92, the article of any of embodiments 3-10 and 87-92, the system of any of embodiments 11, 13-32 and 42-92, or the method of any of embodiments 12-31, 33-44 and 56-92, wherein the graphic is in the form of a sticker.

94. The graphic of any of embodiments 1, 2 and 87-93, the article of any of embodiments 3-10 and 87-93, the system of any of embodiments 11, 13-32 and 42-93, or the method of any of embodiments 12-31, 33-44 and 56-93, wherein the representation of an anatomical feature is a first feature of the graphic, and wherein the graphic further includes a second feature that is non-anatomical.

95. The graphic, article, system or method of embodiment 94, wherein the second feature is located adjacent the first feature.

96. The graphic, article, system or method of embodiment 94 or 95, wherein the second feature is positioned relative to the first feature to provide additional guidance for coupling the medical article securement system to a subject in a desired orientation relative to the subject.

97. The graphic, article, system or method of any of embodiments 94-96, wherein the second feature includes a directional cue.

98. The graphic, article, system or method of embodiment 97, wherein the directional cue includes an arrow.

The embodiments described above and illustrated in the figures are presented by way of example only and are not intended as a limitation upon the concepts and principles of the present disclosure. As such, it will be appreciated by one having ordinary skill in the art that various changes in the elements and their configuration and arrangement are possible without departing from the spirit and scope of the present disclosure.

All references and publications cited herein are expressly incorporated herein by reference in their entirety into this disclosure.

Various features and aspects of the present disclosure are set forth in the following claims.

1-3. (canceled)

4. A medical article securement system, the system comprising:
a graphic comprising a representation of an anatomical feature, such that the graphic provides a visual cue for coupling the medical article securement system to a subject in a desired orientation relative to the subject;
the system configured to be coupled to the subject and further configured to secure at least a portion of a medical article.

5. A method for coupling a medical article securement system to a subject, the method comprising:
providing a medical article securement system comprising a graphic, the graphic comprising a representation of an anatomical feature, such that the graphic provides a visual cue for coupling the medical article securement system to a subject in a desired orientation relative to the subject; and
coupling the medical article securement system to the subject according to the graphic.

6. The system of claim 4, wherein the medical article securement system has a first end and a second end, and wherein the graphic is located toward the first end to indicate that the first end of the medical article securement system is to be oriented toward the anatomical feature of the subject that is represented by the graphic.

7. The system of claim 4, wherein at least a portion of the medical article securement system is shaped to provide at least a portion of the graphic.

8. The system of claim 4, wherein the representation of an anatomical feature is a first feature of the graphic, and further comprising a second feature that is non-anatomical, and wherein at least a portion of the medical article securement system is shaped into at least one of the first feature and the second feature.

9. The system of claim 4, wherein the medical article securement system includes a bracket configured to secure a medical article, and wherein the bracket includes at least a portion of the graphic.

10. The system of claim 9, wherein the bracket is shaped into at least a portion of the graphic.

11. The system of claim 4, wherein the medical article securement system includes a disposable portion, wherein the disposable portion includes a liner, a carrier, a frame, or a combination thereof, and wherein the disposable portion includes at least a portion of the graphic.

12. The system of claim 4, wherein the medical article securement system includes a base dressing configured to be coupled to the subject, and wherein the base dressing includes at least a portion of the graphic.

13. The system of claim 12, wherein the base dressing is shaped to provide at least a portion of the graphic.

14. The system of claim 4, wherein the graphic has an optical property that is different from at least an adjacent portion of the medical article securement system.

15. The system of claim 4, wherein the graphic includes a cutout from a portion of the medical article securement system.

16-17. (canceled)

18. The system of claim 4, wherein the anatomical feature includes at least one of a whole body, an upper body, a lower body, and a body part.

19. The system of claim 4, wherein the anatomical feature includes at least a portion of at least one of a limb, a hand, a foot, a finger, a toe, a head, a neck, a torso, an internal organ, and a combination thereof.

20. The system of claim 4, wherein the anatomical feature includes at least a portion of at least one of a heart, a stomach, a bladder, a brain, a lung, a skeletal feature, and a combination thereof.

21. The system of claim 4, wherein the representation of an anatomical feature includes at least one of an outline, a cartoon, an anatomically-accurate graphic, and a combination thereof.

22. (canceled)

23. The system of claim 4, wherein the graphic is in the form of a sticker.

24-25. (canceled)
